(19) United States
(12) Patent Application Publication
(10) Pub. No.: US 2007/0225499 A1
(43) Pub. Date: Sep. 27, 2007

(54) Process for the Preparation of 2,2-Disubstituted Pyrroles

(76) Inventors: Gary Javadi, West Windsor, NJ (US); Sandor Karady, Mountainside, NJ (US); Kenji Maeda, Okazaki (JP); Ross A. Miller, Fanwood, NJ (US); Ronald H. Szumigala, Somerville, NJ (US)

Correspondence Address: MERCK AND CO., INC, P O BOX 2000, RAHWAY, NJ, 07065-0907, US

(21) Appl. No.: 11/578,756

(22) PCT Filed: Apr. 15, 2005
(86) PCT No.: PCT/US05/13630

§ 371 Date: Oct. 18, 2006

Related U.S. Application Data
(60) Provisional application No. 60/563,583, filed on Apr. 19, 2004.

(51) Int. Cl. C07D 207/16; C07D 211/58; C07D 263/04; C07D 263/52

(52) U.S. Cl. 546/208; 548/221; 548/565

(57) ABSTRACT

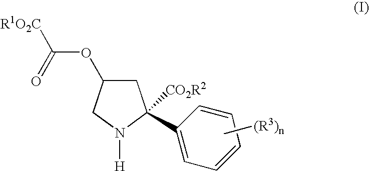

The present invention relates to the stereoselective preparation of 2,2-disubstituted-4-carbonatepyrroles from readily available chiral starting materials. Such pyrroles are useful as intermediates in the preparation of 2,2,4-trisubstituted 2,5-dihydropyrroles, that are inhibitors of mitotic kinesins and are useful for treating cellular proliferative diseases, for treating disorders associated with KSP kinesin activity, and for inhibiting KSP kinesin. The product of the process of the invention may be illustrated by the Formula (I).

### BACKGROUND OF THE INVENTION

This invention relates to the stereoselective preparation of 2,2-disubstituted-4-carbonatepyrroles useful as intermediates in the syntheses of inhibitors of mitotic kinesins useful in the treatment of cellular proliferative diseases, for example cancer.

Among the therapeutic agents used to treat cancer are the taxanes and vinca alkaloids. Taxanes and vinca alkaloids act on microtubules, which are present in a variety of cellular structures. Microtubules are the primary structural element of the mitotic spindle. The mitotic spindle is responsible for distribution of replicate copies of the genome to each of the two daughter cells that result from cell division. It is presumed that disruption of the mitotic spindle by these drugs results in inhibition of cancer cell division, and induction of cancer cell death. However, microtubules form other types of cellular structures, including tracks for intracellular transport in nerve processes. Because these agents do not specifically target mitotic spindles, they have side effects that limit their usefulness.

Improvements in the specificity of agents used to treat cancer is of considerable interest because of the therapeutic benefits which would be realized if the side effects associated with the administration of these agents could be reduced. Traditionally, dramatic improvements in the treatment of cancer are associated with identification of therapeutic agents acting through novel mechanisms. Examples of this include not only the taxanes, but also the camptothecin class of topoisomerase I inhibitors. From both of these perspectives, mitotic kinesins are attractive targets for new anti-cancer agents.

Mitotic kinesins are enzymes essential for assembly and function of the mitotic spindle, but are not generally part of other microtubule structures, such as in nerve processes. Mitotic kinesins play essential roles during all phases of mitosis. These enzymes are “molecular motors” that transform energy released by hydrolysis of ATP into mechanical force which drives the directional movement of cellular cargoes along microtubules. The catalytic domain sufficient for this task is a compact structure of approximately 340 amino acids. During mitosis, kinesins organize microtubules into the bipolar structure that is the mitotic spindle. Kinesins mediate movement of chromosomes along spindle microtubules, as well as structural changes in the mitotic spindle associated with specific phases of mitosis. Experimental perturbation of mitotic kinesin function causes malformation or dysfunction of the mitotic spindle, frequently resulting in cell cycle arrest and cell death.

Among the mitotic kinesins which have been identified is KSP. KSP belongs to an evolutionarily conserved kinesin subfamily of plus end-directed microtubule motors that assemble into bipolar homotetramers consisting of antiparallel homodimers. During mitosis KSP associates with microtubules of the mitotic spindle. Microinjection of antibodies directed against KSP into human cells prevents spindle pole separation during prometaphase, giving rise to monopolar spindles and causing mitotic arrest and induction of programmed cell death. KSP and related kinesins in other, non-human, organisms, bundle antiparallel microtubules and slide them relative to one another, thus forcing the two spindle poles apart. KSP may also mediate in anaphase B spindle elongation and focussing of microtubules at the spindle pole. Human KSP (also termed HsEg5) has been described Disubstituted and trisubstituted dihydropyrroles have recently been described as being inhibitors of KSP (PCT Publ. WO 03/105855, Dec. 24, 2003).

Mitotic kinesins are attractive targets for the discovery and development of novel mitotic chemotherapeutics. In light of the discovery that certain 2,2-disubstituted-2,5-dihydropyrroles are potent inhibitors of KSP, it is an object of the present invention to provide high yielding stereoselective syntheses of intermediate compounds in the synthesis of such dihydropyrrole compounds.

### SUMMARY OF THE INVENTION

The present invention relates to the stereoselective preparation of 2,2-disubstituted-4-carbonatepyrroles from readily available chiral starting materials. Such pyrroles are useful as intermediates in the preparation of 2,2,4-trisubstituted 2,5-dihydropyrroles, that are inhibitors of mitotic kinesins and are useful for treating cellular proliferative diseases, for treating disorders associated with KSP kinesin activity, and for inhibiting KSP kinesin. The product of the process of the invention may be illustrated by the Formula I:

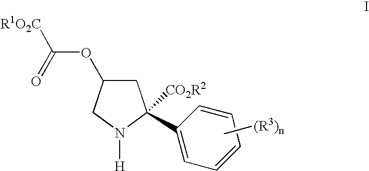

### BRIEF DESCRIPTION OF THE DRAWINGS

FIG. 1 Thermal Analysis of (3R,4S)-3-Fluoro-N,1-dimethylpiperidin-4-amine dihydrochloride (3-5) Form 1 The weight loss curve is shown as a solid line. The dashed line shows the mass spectrum analysis of the volatile produced by the weight loss.

FIG. 2 X-ray Powder Diffraction Pattern of (3R,4S)-3-Fluoro-N,1-dimethylpiperidin-4-amine dihydrochloride (3-5) Form 1

### DETAILED DESCRIPTION OF THE INVENTION

The first aspect of instant invention is directed to a process for the preparation of a compound of Formula I: 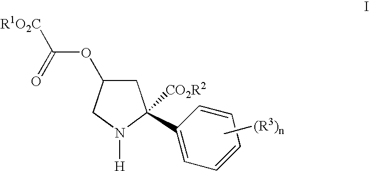
or a salt thereof,
wherein:
a is 0 or 1;
b is 0 or 1;
m is 0, 1, or 2;
n is 1 or 2;
r is 0 or 1;
s is 0 or 1;
R¹ and R² are independently selected from: (C₁-C₆)alkyl, aryl, heterocyclyl and (C₃-C₆)cycloalkyl, optionally substituted with one, two or three substituents selected from R⁴;
R³ is selected from:
1) hydrogen;
2) (C═O)ₐO_{b}C₁-C₁₀ alkyl,
3) (C═O)ₐO_{b}aryl,
4) CO₂H,
5) halo,
6) CN,
7) OH,
8) O_{b}C₁-C₆ perfluoroalkyl,
9) Oₐ(C═O)_{b}NR⁵R⁶,
10) S(O)ₘR^{a},
11) S(O)₂NR⁵R⁶, and
12) —OPO(OH)₂;
   said alkyl and aryl, optionally substituted with one, two or three substituents selected from R⁴;
   R⁴ is selected from:
1) (C═O)ᵣOₛ(C₁-C₁₀)alkyl,
2) Oᵣ(C₁-C₃)perfluoroalkyl,
3) oxo,
4) OH,
5) halo,
6) CN,
7) (C₂-C₁₀)alkenyl,
8) (C₂-C₁₀)alkynyl,
9) (C═O)ᵣOₛ(C₃-C₆)cycloalkyl,
10) (C═O)ᵣOₛ(C₀-C₆)alkylene-aryl,
11) (C═O)ᵣOₛ(C₀-C₆)alkylene-heterocyclyl,
12) (C═O)ᵣOₛ(C₀-C₆)alkylene-N(R^{b})₂,
13) C(O)R^{a},
14) (C₀-C₆)alkylene-CO₂R^{a},
15) C(O)H,
16) (C₀-C₆)alkylene-CO₂H, and
17) (C═O)ᵣN(R^{b})₂,
18) S(O)ₘR^{a},
19) S(O)₂N(R^{b})₂; and
20) —OPO(OH)₂;
   said alkyl, alkenyl, alkynyl, cycloalkyl, aryl, alkylene and heterocyclyl is optionally substituted with up to three substituents selected from R^{b}, OH, (C₁-C₆)alkoxy, halogen, CO₂H, CN, O(C═O)C₁-C₆ alkyl, oxo, NO₂ and N(R^{b})₂;
   R⁵ and R⁶ are independently selected from:
1) H,
2) (C═O)O_{b}C₁-C₁₀ alkyl,
3) (C═O)O_{b}C₃-C₈ cycloalkyl,
4) (C═O)O_{b}aryl,
5) (C═O)O_{b}heterocyclyl,
6) C₁-C₁₀ alkyl,
7) aryl,
8) C₂-C₁₀ alkenyl,
9) C₂-C₁₀ alkynyl,
10) heterocyclyl,
11) C₃-C₈ cycloalkyl,
12) SO₂R^{a}, and
13) (C═O)NR^{b}₂,
   said alkyl, cycloalkyl, aryl, heterocylyl, alkenyl, and alkynyl is optionally substituted with one, two or three substituents selected from R⁴, or
   R⁵ and R⁶ can be taken together with the nitrogen to which they are attached to form a monocyclic or bicyclic heterocycle with 3-7 members in each ring and optionally containing, in addition to the nitrogen, one or two additional heteroatoms selected from N, O and S, said monocyclic or bicyclic heterocycle optionally substituted with one, two or three substituents selected from R⁴;
   R^{a} is independently selected from: (C₁-C₆)alkyl, (C₃-C₆)cycloalkyl, aryl, or heterocyclyl, optionally substituted with one, two or three substituents selected from R⁴;
   R^{b} is independently selected from: H, (C₁-C₆)alkyl, aryl, heterocyclyl, (C₃-C₆)cycloalkyl, (C═O)OC₁-C₆ alkyl, (C═O)C₁-C₆ alkyl, (C═O)aryl, (C═O)heterocyclyl, (C═O)NR^{e}R^{e′} or S(O)₂R^{a}, optionally substituted with one, two or three substituents selected from R⁴;
   which comprises the step of reacting a compound of the formula II: 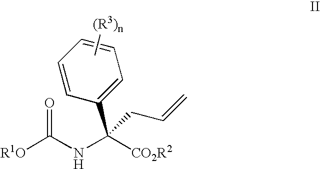
   with a halogenating agent in an aqueous solvent to produce the compound of formula I.

In an embodiment of the process of the instant invention, R¹ and R² are independently selected from: (C₁-C₆)alkyl.

In another embodiment of the process of the instant invention, R¹ and R² are independently selected from: methyl and ethyl.

In an embodiment of the process of the first aspect of the instant invention, the aqueous solvent is selected from: an acetonitrile/water mixture, a tetrahydrofuran/water mixture and a isopropyl acetate/water mixture. In a further embodiment, the aqueous solvent is an acetonitrile/water mixture.

In an embodiment of the process of the first aspect of the instant invention, the halogenating agent is iodine (I₂).

In the second aspect of the process of the instant invention, the process for preparing the compound of the formula I, or a salt thereof, described above further comprises the steps of
a) reacting the compound of the formula III: 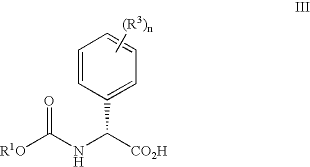
with a benzaldehyde source, in the presence of an acid to produce the compound of the formula IV: 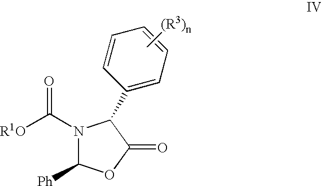
and b) converting the compound of the formula IV to the compound of formula II;
wherein R³ is as described above.

In an embodiment of the process of the second aspect of the instant invention, the acid is selected from: phenyl sulfonic acid,

In an embodiment of the process of the second aspect of the instant invention, the benzaldehyde source is benzaldehyde dimethyl acetal.

In an embodiment of the process of the second aspect of the instant invention, the conversion of the compound of the formula IV to the compound of formula II comprises the step of adding a base to a solution of a mixture of the compound of the formula IV and an allylating agent. In another embodiment the allylating agent is allyl bromide. In another embodiment the base in this step is sodium bis(trimethylsilyl)amide

In a third aspect of the instant invention, the process described above for preparing the compound of the formula I, or a salt thereof, further comprises the steps of
a) reacting the compound of the formula III: 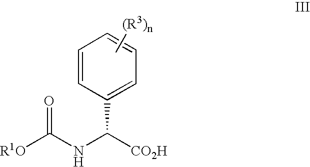
with a benzaldehyde source, in the presence of an acid to produce the compound of the formula IV: 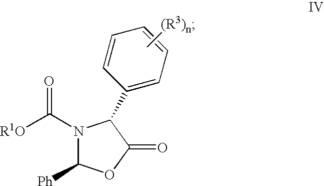
b) crystallizing the compound of formula IV from a crystallization solvent; and
c) converting the compound of the formula IV to the compound of formula II;
wherein R³ is as described above.

In an embodiment of the third aspect of the process of the invention, the benzaldehyde source is benzaldehyde dimethyl acetal.

In an embodiment of the third aspect of the process of the invention, the crystallization solvent is selected from toluene, a toluene/hexanes mixture, a toluene/heptane mixture and a toluene/octane mixture. In a further embodiment of the third aspect of the process of the invention, the crystallization solvent is a toluene/hexanes mixture.

A fourth aspect of the instant invention is directed to the preparation of a compound of the formula V, or a salt thereof: 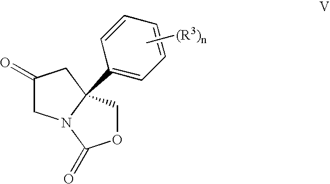
wherein R³ is as described above,
which comprises the steps of:
a) converting the compound of the formula I, as described above, to the compound of the formula VI: 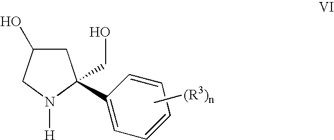
b) reacting the compound of the formula VI with a carbon monoxide diradical source to produce the compound of the formula VII: 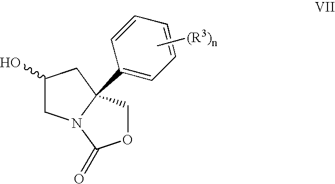
and
c) reacting the compound of the formula VII with an oxidizing agent to product the compound of the formula V.

In an embodiment of the process of the fourth aspect of the instant invention, the conversion of the compound of the formula I to the compound of the formula VI is accomplished by treating the compound of the formula I with a reducing agent. In a further embodiment, the reducing agent is selected from LiBH₄, LiAlH₄, LiH(Ot-Bu)₃, Red-Al® and the like. In another embodiment, the reducing agent is Red-Al®.

In an embodiment of the process of the fourth aspect of the instant invention, the carbon monoxide diradical source is 1,1′-carbonyldiimidazole.

In an embodiment of the process of the fourth aspect of the instant invention, the oxidizing agent is sodium hypochlorite with a catalytic amount of tetrapropylammoniumperruthenate.

Particular compounds of the instant invention are: 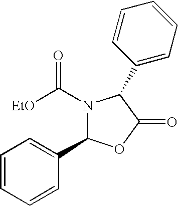
ethyl (2S,4R)-5-oxo-2,4-diphenyl-1,3-oxazolidine-3-carboxylate; 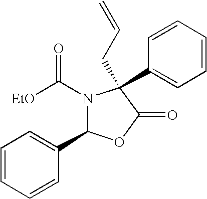
ethyl (2S,4S)-4-allyl-5-oxo-2,4-diphenyl-1,3-oxazolidine-3-carboxylate; 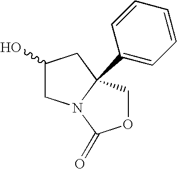
(7aS)-6-hydroxy-7a-phenyltetrahydro-1H-pyrrolo[1,2-c][1,3]oxazol-3-one; and 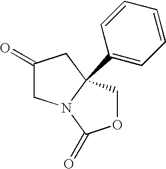
(7aS)-7a-phenyldihydro-1H-pyrrolo[1,2-c][1,3]oxazole-3,6(5H)-dione.

The compounds of the present invention may have asymmetric centers, chiral axes, and chiral planes (as described in: E. L. Eliel and S. H. Wilen, Stereochemistry of Carbon Compounds, John Wiley & Sons, New York, 1994, pages 1119-1190), and occur as racemates, racemic mixtures, and as individual diastereomers, with all possible isomers and mixtures thereof, including optical isomers, all such stereoisomers being included in the present invention. In addition, the compounds disclosed herein may exist as tautomers and both tautomeric forms are intended to be encompassed by the scope of the invention, even though only one tautomeric structure is depicted.

When any variable (e.g. R⁴, R⁷, R¹⁰, etc.) occurs more than one time in any constituent, its definition on each occurrence is independent at every other occurrence. Also, combinations of substituents and variables are permissible only if such combinations result in stable compounds. Lines drawn into the ring systems from substituents represent that the indicated bond may be attached to any of the substitutable ring atoms. If the ring system is polycyclic, it is intended that the bond be attached to any of the suitable carbon atoms on the proximal ring only.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be taken to be equivalent to the phrase “optionally substituted with at least one substituent” and in such cases the preferred embodiment will have from zero to three substituents.

As used herein, “alkyl” is intended to include both branched and straight-chain saturated aliphatic hydrocarbon groups having the specified number of carbon atoms. For example, C₁-C₁₀, as in “C₁-C₁₀ alkyl” is defined to include groups having 1, 2, 3, 4, 5, 6, 7, 8, 9 or 10 carbons in a linear or branched arrangement. For example, “C₁-C₁₀ alkyl” specifically includes methyl, ethyl, n-propyl, i-propyl, n-butyl, t-butyl, i-butyl, pentyl, hexyl, heptyl, octyl, nonyl, decyl, and so on. The term “cycloalkyl” means a monocyclic saturated aliphatic hydrocarbon group having the specified number of carbon atoms. For example, “cycloalkyl” includes cyclopropyl, methyl-cyclopropyl, 2,2-dimethyl-cyclobutyl, 2-ethyl-cyclopentyl, cyclohexyl, and so on. In an embodiment of the invention the term “cycloalkyl” includes the groups described immediately above and further includes monocyclic unsaturated aliphatic hydrocarbon groups. For example, “cycloalkyl” as defined in this embodiment includes cyclopropyl, methyl-cyclopropyl, 2,2-dimethyl-cyclobutyl, 2-ethyl-cyclopentyl, cyclohexyl, cyclopentenyl, cyclobutenyl and so on.

The term “alkylene” means a hydrocarbon diradical group having the specified number of carbon atoms. For example, “alkylene” includes —CH₂—, —CH₂CH₂— and the like.

When used in the phrases “C₁-C₆ aralkyl” and “C₁-C₆ heteroaralkyl” the term “C₁-C₆” refers to the alkyl portion of the moiety and does not describe the number of atoms in the aryl and heteroaryl portion of the moiety.

“Alkoxy” represents either a cyclic or non-cyclic alkyl group of indicated number of carbon atoms attached through an oxygen bridge. “Alkoxy” therefore encompasses the definitions of alkyl and cycloalkyl above.

If no number of carbon atoms is specified, the term “alkenyl” refers to a non-aromatic hydrocarbon radical, straight, branched or cyclic, containing from 2 to 10 carbon atoms and at least one carbon to carbon double bond. Preferably one carbon to carbon double bond is present, and up to four non-aromatic carbon-carbon double bonds may be present. Thus, “C₂-C₆ alkenyl” means an alkenyl radical having from 2 to 6 carbon atoms. Alkenyl groups include ethenyl, propenyl, butenyl, 2-methylbutenyl and cyclohexenyl. The straight, branched or cyclic portion of the alkenyl group may contain double bonds and may be substituted if a substituted alkenyl group is indicated.

The term “alkynyl” refers to a hydrocarbon radical straight, branched or cyclic, containing from 2 to 10 carbon atoms and at least one carbon to carbon triple bond. Up to three carbon-carbon triple bonds may be present. Thus, “C₂-C₆ alkynyl” means an alkynyl radical having from 2 to 6 carbon atoms. Alkynyl groups include ethynyl, propynyl, butynyl, 3-methylbutynyl and so on. The straight, branched or cyclic portion of the alkynyl group may contain triple bonds and may be substituted if a substituted alkynyl group is indicated.

In certain instances, substituents may be defined with a range of carbons that includes zero, such as (C₀-C₆)alkylene-aryl. If aryl is taken to be phenyl, this definition would include phenyl itself as well as —CH₂Ph, —CH₂CH₂Ph, CH(CH₃)CH₂CH(CH₃)Ph, and so on.

As used herein, “aryl” is intended to mean any stable monocyclic or bicyclic carbon ring of up to 7 atoms in each ring, wherein at least one ring is aromatic. Examples of such aryl elements include phenyl, naphthyl, tetrahydronaphthyl, indanyl and biphenyl. In cases where the aryl substituent is bicyclic and one ring is non-aromatic, it is understood that attachment is via the aromatic ring.

The term heteroaryl, as used herein, represents a stable monocyclic or bicyclic ring of up to 7 atoms in each ring, wherein at least one ring is aromatic and contains from 1 to 4 heteroatoms selected from the group consisting of O, N and S. Heteroaryl groups within the scope of this definition include but are not limited to: acridinyl, carbazolyl, cinnolinyl, quinoxalinyl, pyrrazolyl, indolyl, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. As with the definition of heterocycle below, “heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring, respectively.

The term “heterocycle” or “heterocyclyl” as used herein is intended to mean a 5- to 10-membered aromatic or nonaromatic heterocycle containing from 1 to 4 heteroatoms selected from the group consisting of O, N and S, and includes bicyclic groups. “Heterocyclyl” therefore includes the above mentioned heteroaryls, as well as dihydro and tetrathydro analogs thereof. Further examples of “heterocyclyl” include, but are not limited to the following: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrahydrothiopyranyl, tetrahydroisoquinolinyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof. Attachment of a heterocyclyl substituent can occur via a carbon atom or via a heteroatom.

Preferably, heterocycle is selected from 2-azepinone, benzimidazolyl, 2-diazapinone, imidazolyl, 2-imidazolidinone, indolyl, isoquinolinyl, morpholinyl, piperidyl, piperazinyl, pyridyl, pyrrolidinyl, 2-piperidinone, 2-pyrimidinone, 2-pyrollidinone, quinolinyl, tetrahydrofuryl, tetrahydroisoquinolinyl, and thienyl.

As appreciated by those of skill in the art, “halo” or “halogen” as used herein is intended to include chloro, fluoro, bromo and iodo.

The alkyl, alkenyl, alkynyl, cycloalkyl, aryl, heteroaryl and heterocyclyl substituents may be substituted or unsubstituted, unless specifically defined otherwise. For example, a (C₁-C₆)alkyl may be substituted with one, two or three substituents selected from OH, oxo, halogen, alkoxy, dialkylamino, or heterocyclyl, such as morpholinyl, piperidinyl, and so on. In this case, if one substituent is oxo and the other is OH, the following are included in the definition: —C═O)CH₂CH(OH)CH₃, —(C═O)OH, —CH₂(OH)CH₂CH(O), and so on.

In certain instances, R⁵ and R⁶ are defined such that they can be taken together with the nitrogen to which they are attached to form a monocyclic or bicyclic heterocycle with 5-7 members in each ring and optionally containing, in addition to the nitrogen, one or two additional heteroatoms selected from N, O and S, said heterocycle optionally substituted with one or more substituents selected from R⁴. Examples of the heterocycles that can thus be formed include, but are not limited to the following, keeping in mind that the heterocycle is optionally substituted with one or more (and in an embodiment, one, two or three) substituents chosen from R⁴:

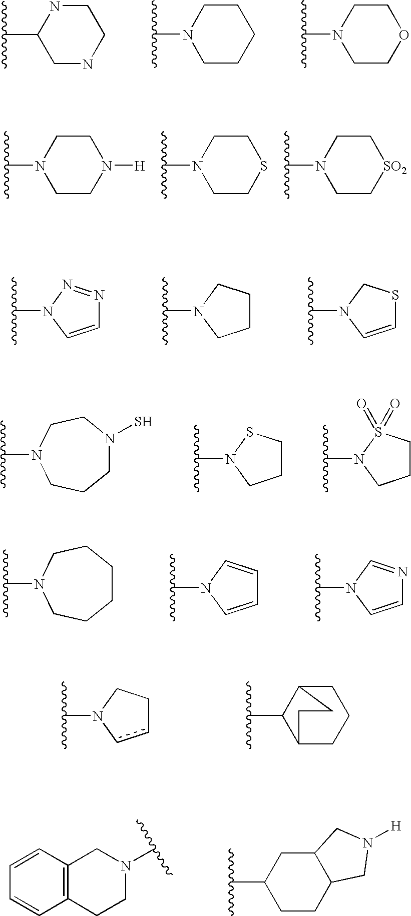

In an embodiment, R¹ is selected from C₁-C₆ alkyl. In a further embodiment, R¹ is ethyl.

In an embodiment, R² is selected from C₁-C₆ alkyl. In a further embodiment, R² is methyl.

In an embodiment, R³ is selected from H, —OH, halogen and C₁-C₆ alkyl.

Aqueous solvents useful in the process of the first aspect of the invention include, but are not limited to: an acetonitrile/water mixture, a tetrahydrofuran/water mixture and a isopropyl acetate/water mixture.

Halogenating agents useful in the process of the first aspect of the invention include, but are not limited to, iodine (I₂), bromine (Br₂), dibromodimethylhydantoin, N-bromosuccinamide, N-iodosuccinamide, iodine monochloride and the like.

Acids useful in the process of the second and third aspects of the invention may be illustrated as HL, wherein L- is selected from the group consisting of:
(1) halide,
(2) cyamide,
(3) BF₄—,
(4) (C₆F₅)₄B—,
(5) MF₆—, wherein M is P, As, or Sb,
(6) ClO₄—,
(7) benzotriazolyl anion,
(8) aryl-SO₃—, wherein the aryl is optionally substituted with one or more substituents each of which is independently halo, C₁-C₁₀ alkyl, or C₁-C₁₀ haloalkyl,
(9) C₁-C₆ alkyl-SO₃— wherein the alkyl is optionally substituted with one or more halogens, and
(10) trihaloacetate anion.

In another embodiment of the process of the second and third aspects of the invention, L-, of the acid HL, is selected from the group consisting of fluoride, chloride, cyamide, BF₄—, (C₆F₅)₄B—, PF₆—, ClO₄—, benzotriazolyl anion, OTf-, CF₃CF₂SO₃—, C₆F₅SO₃—, OTs-, and CF₃CO₂—.

In still another embodiment of the process of the second and third aspects of the invention, L-, of the acid HL, is a weakly nucleophilic or non-nucleophilic anion. Stated alternatively, L- in this embodiment is a very weak base and when L is attached to carbon, L can be readily displaced as L- by a variety of nucleophiles. In an aspect of this embodiment, L-, of the acid HL, is selected from the group consisting of fluoride, chloride, BF₄—, (C₆F₅)₄B—, PF₆—, AsF₆—, SbF₆—, ClO₄—, benzotriazolyl anion, OTf-, CF₃CF₂SO₃—, C₆F₅SO₃—, OTs-, and CF₃CO₂—.

Benzaldehyde sources useful in the process of the second and third aspects of the invention include, but are not limited to benzaldehyde dimethyl acetal, benzaldehyde, diethoxy or isopropoxybenzaldehyde, diacetoxybenzaldehyde, and the like.

Crystallization solvents useful in the third aspect of the process of the invention include, but are not limited to, toluene, hexanes, heptane, octane, a toluene/hexanes mixture, a toluene/heptane mixture, a toluene/octane mixture, benzene, methyl t-butylether, and the like. It is understood that additional mixtures or combinations of the listed solvents may also be useful for the described crystallization.

Carbon monoxide diradical sources useful in the process of the fourth aspect of the instant invention, include, but are not limited to: 1,1′-carbonyldiimidazole, phosgene, triphosgene and the like.

Allylating agents useful in the process of the second aspect of the instant invention, include, but are not limited to: allyl chloride, allyl bromide and the like. Bases useful in the process of the second aspect of the instant invention, include, but are not limited to: sodium bis(trimethylsilyl)amide, and the like.

Oxidizing agents useful in the process of the fourth aspect of the instant invention, include, but are not limited to: nitroxyl radicals, MCPBA, chlorinating reagents (such as trichloroisocyanuric acid, N-chlorosuccinimide, chlorine, calcium hypochlorite, sodium hypochlorite and the like), ozone, sodium bromite, metal salts (such as potassium dichromate, sodium dichromate, potassium permanganate, sodium permanganate and the like), [bis(acetoxy)iodo]benzene, electrooxidation and stoichiometric oxoamminium salts. Such oxidizing agents can be used alone or in combination with an oxidation catalyst, which includes, but is not limited to: 2,2,6,6-trimethyl-1-piperidinyloxy, free radical, tetrapropylammoniumperruthenate (TPAP), ruthenium trichloride, ruthenium oxide and the like. When an oxidizing agent and an oxidation catalyst are used in combination, the combination itself is referred to as an oxidizing agent. Additional oxidizing agents are comprehensively listed in R. C. Larock Comprehensive organic transformations 2^{nd} Edition (1999), pages 1235-1249.

Included in the instant invention is the free form of compounds whose syntheses is described, as well as the salts thereof. The term “free form” refers to the amine compounds in non-salt form. The encompassed salts not only include the salts exemplified for the specific compounds described herein, but also all the typical salts of those compounds. The free form of the specific salt compounds described may be isolated using techniques known in the art. For example, the free form may be regenerated by treating the salt with a suitable dilute aqueous base solution such as dilute aqueous NaOH, potassium carbonate, ammonia and sodium bicarbonate. The free forms may differ from their respective salt forms somewhat in certain physical properties, such as solubility in polar solvents.

The salts of the compounds prepared by the processes of the instant invention are those of the compounds of this invention which contain a basic or acidic moiety. Generally, the salts of basic compounds are prepared either by ion exchange chromatography or by reacting the free base with stoichiometric amounts or with an excess of the desired salt-forming inorganic or organic acid in a suitable solvent or various combinations of solvents. Similarly, the salts of acidic compounds are formed by reactions with the appropriate inorganic or organic base.

Thus, salts of the basic compounds prepared by the processes of this invention include the conventional non-toxic salts such as those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like.

When the compound prepared by the processes of the present invention is acidic, salt refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine caffeine, choline, N,N¹-dibenzylethylenediamine, diethylamin, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

It will also be noted that the compounds of the present invention are potentially internal salts or zwitterions, since under physiological conditions a deprotonated acidic moiety in the compound, such as a carboxyl group, may be anionic, and this electronic charge might then be balanced off internally against the cationic charge of a protonated or alkylated basic moiety, such as a quaternary nitrogen atom.

The following abbreviations, used in the Schemes and Examples, are defined below:

| | |
|---|---|
| CDI | 1,1′-carbonyldiimidazole |
| CSP HPLC | Chiral stationary phase high performance liquid |
| | chromatography |
| DAST | (diethylamino) sulfur trifluoride |
| DCE | 1,2-dichloroethane |
| DCM | Dichloromethane |
| DMF | Dimethylformamide |
| DMSO | Dimethyl sulfoxide |
| EtOAc | Ethyl acetate |
| IPAC | Isopropyl acetate |
| LAH | Lithium aluminum hydride |
| LiHMDS | Lithium hexamethyldisilazide |
| MsCl | Methanesulfonylchloride |
| NaHMDS | Sodium hexamethyldisilazide |
| NOE | Nuclear Overhauser Effect |
| PTC | Phase transfer catalyst |
| TBSCl | tert-butyldimethylsilyl chloride |
| TEA | Triethylamine |
| TFA | Trifluoroacetic acid |
| THF | Tetrahydrofuran |
| TPAP | Tetrapropylammoniumperruthenate |

The processes of this invention may be employed as generally shown in the following schemes, in addition to other standard manipulations that are known in the literature or exemplified in the experimental procedures. The illustrative schemes below, therefore, are not limited by the chemical reagents listed or by any particular substituents employed for illustrative purposes. Substituent numbering as shown in the schemes does not necessarily correlate to that used in the claims and often, for clarity, a single substituent is shown attached to the compound where multiple substituents are allowed under the definitions of Formula I hereinabove.

### Schemes

As shown in Scheme A, key pyrrolo[1,2-c][1,3]oxazol-3,6(5H)-dione intermediate A-9 may be obtained from readily available suitably substituted (S) α-phenylglycines. Carbonate protection of the amine, followed by reductive cyclization with a benzaldehyde source, such as the acetal illustrated, stereoselectively provides the oxazolinone A-3 Ring allylation provides intermediate A-4, which then undergoes saponification to provide the α,α-disubstituted glycinate A-5. Halogen-mediated cyclization of A-5 is accompanied by unexpected migration of the carbonate to the hydroxyl moiety to provide pyrrole A-6. Reductive cleavage of the carbonate group and cyclization with a carbon monoxide diradical (such as CDI as shown, provides A-9.

Scheme B illustrates conversion of A-9 to a 2,2,4-trisubstituted dihydropyrrole B-2. The dihydropyrrole may be utilized directly as illustrated in PCT Publication WO 03/105855 to provide potent inhibitors of the mitotic kinesin KSP. Alternatively, B-2 may be treated with triphosgene to provide the intermediate B-3, which can be reacted with a variety of suitably substituted amines, as shown in Schemes C and D, to provide such mitotic kinesin inhibitors.

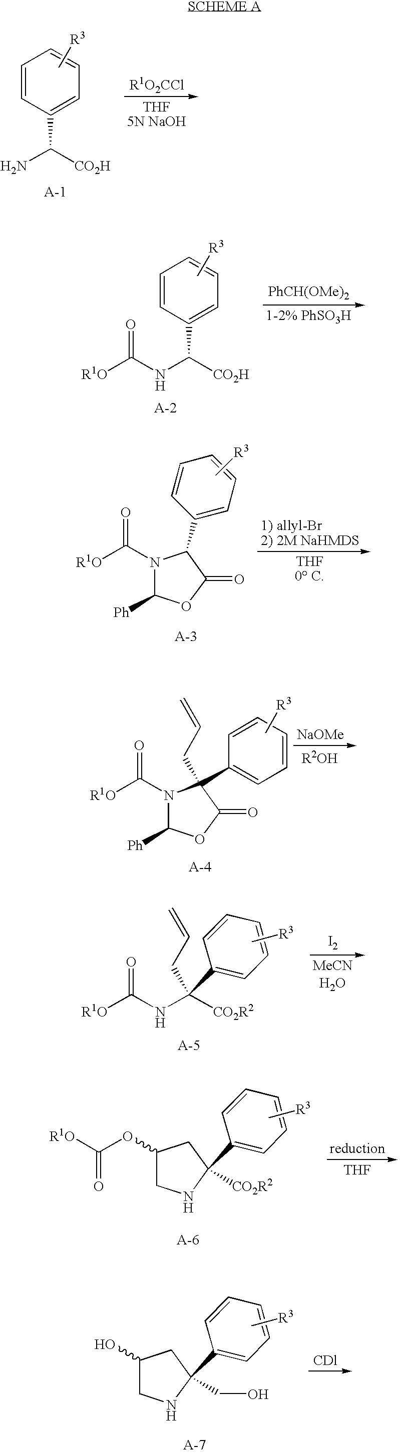

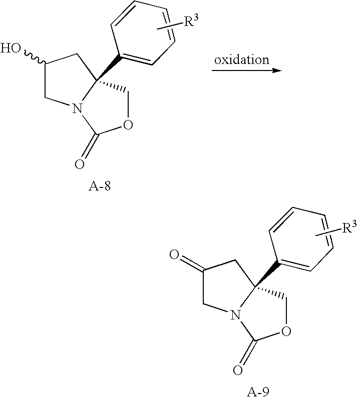

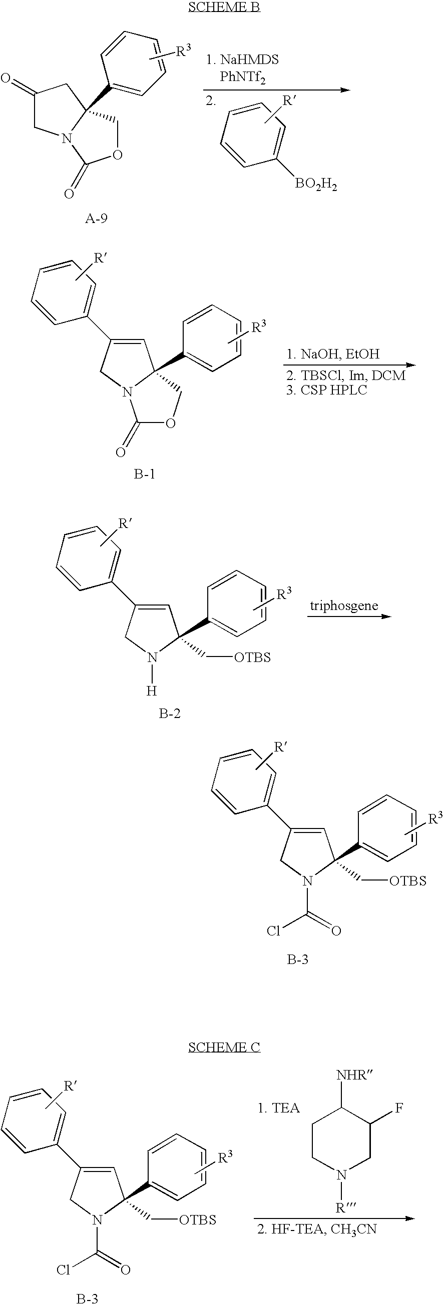

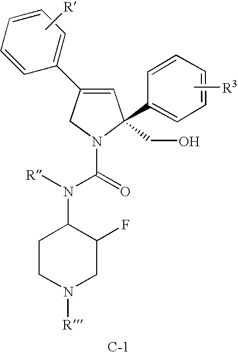

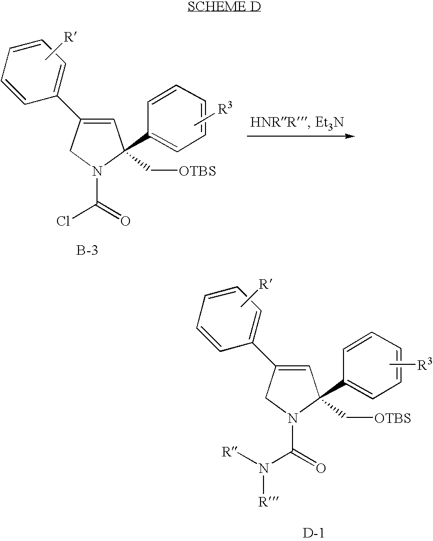

### EXAMPLES

Examples provided are intended to assist in a further understanding of the invention. Particular materials employed, species and conditions are intended to be illustrative of the invention and not limiting of the reasonable scope thereof.

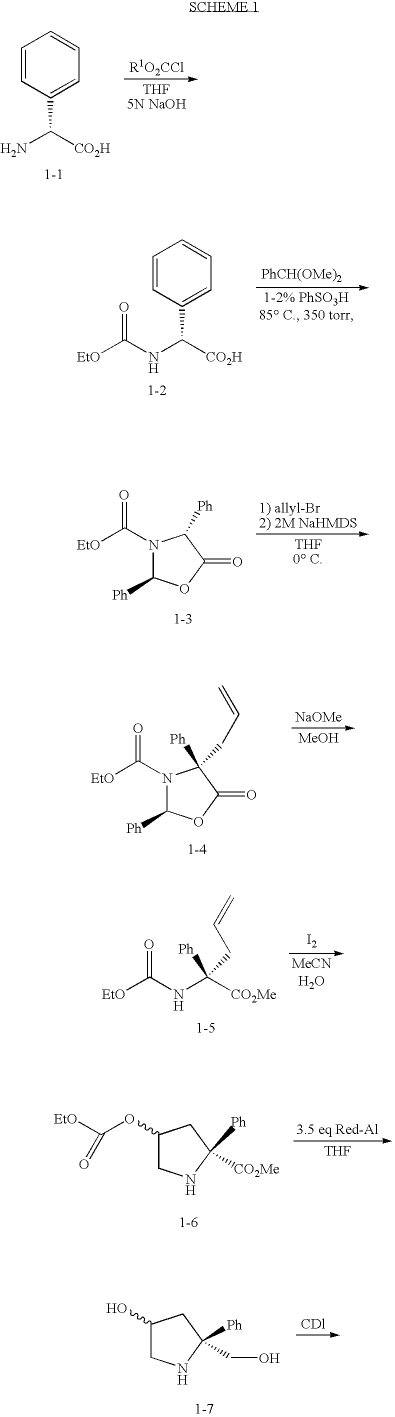

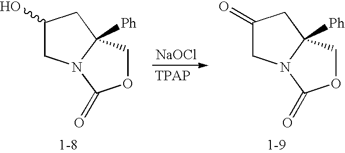

### Step 1: (2R)-[(ethoxycarbonyl)amino](phenyl)acetic acid (1-2)

To a 0° C. mixture of (R)-(−)-2-phenylglycine (1-1, 4 kg) in THF and 5N NaOH (10.6 L) was added ethyl chloroformate over 1 h with the internal temperature maintained below 10° C. Upon completion of the addition, the reaction was aged for 15 min at 0-10° C. and assayed for completion. The reaction was quenched with 37% HCl (until pH=1, 2.3 L) with the internal temperature maintained <25° C. Toluene (20 L) was added and after agitation/settling, the aqueous layer was cut. The organic layer was assayed for yield and solvent switched to toluene. The slurry of 1-2 was used directly in the next reaction. (2R)-[(ethoxycarbonyl)amino]-(phenyl)acetic acid: mp 154-156° C.; ¹H NMR (CDCl₃, 400 MHz) indicated a 1.1:1 mixture of rotamers: δ=12.12 (bs, 2H), 7.99 (d, J=5.3 Hz, 1H), 7.45-7.32 (m, 10H), 5.78 (d, J=6.2 Hz, 1H), 5.41 (d, J=7.1 Hz, 1H), 5.25 (d, J=5.7 Hz, 1H), 4.12 (m, 2H), 4.05 (m, 2H), 1.24 (t, J=6.9 Hz, 3H), 1.06 (t, J=7.0 Hz, 3H); ¹³C NMR (CDCl₃, 100 MHz): δ=175.1, 173.6, 157.3, 155.8, 137.4, 136.1, 129.0, 128.7, 128.6, 128.2, 127.2, 127.1, 62.1, 61.5, 58.3, 57.7, 14.4, 14.1; MS m/z 224 ([M+H]⁺, C₁₁H₁₄NO₄, calc'd 224.09).

### Step 2: ethyl (2S,4R)-5-oxo-2,4-diphenyl-1,3-oxazolidine-3-carboxylate (1-3)

To an 85° C. solution of 1-2 and PhSO₃H (42.7 gm) in toluene under reduced pressure (350 torr), was added a solution of benzaldehyde dimethyl acetal (3 L) in toluene (5 mL/g) over 1-2 h. Toluene/MeOH was distilled off through the course of reaction. Upon completion of the reaction, the solution was cooled to rt and diluted with THF (36 L), until homogeneous. The organic solution was washed with 10% NaHSO₃ (7.5 L), followed by sat'd. NaHCO₃ (9 L). The solvent was then switched to toluene and diluted to 7.5 mL/g total volume (vs. assay yield) with toluene upon completion. The slurry was heated to 75° C. and aged until homogeneous. Upon slow cooling, 1-3 crystallized. When the slurry reached 40° C., heptane (2.5 mL/g) was added. The slurry was cooled to rt and filtered to collect the solid. The solid washed with 1:1 toluene/heptane (5 mL/g) and dried to a constant weight under a nitrogen stream. ethyl (2S,4R)-5-oxo-2,4-diphenyl-1,3-oxazolidine-3-carboxylate: mp 197-199° C.; ¹H NMR (CDCl₃, 400 Hz) δ=7.46-7.37 (m, 10H), 6.77 (bs, 1H), 5.45 (bs, 1H), 3.96 (m, 2H), 3.86 (m, 2H), 0.84 (t, J=7.1 Hz, 3H); ¹³C NMR (CDCl₃, 100 MHz): δ=130.2, 129.1, 129.0, 218.8, 126.7, 90.3, 61.9, 60.3, 13.8; MS m/z 312 ([M+H]⁺, C₁₈H₁₈NO₄, calc'd 312.12).

### Step 3: ethyl (2S,4S)-4-allyl-5-oxo-2,4-diphenyl-1,3-oxazolidine-3-carboxylate (1-4)

To an −10° C. solution of 1-3 and allyl-Br (1.67 L) in THF (40 L) was added a 2M solution of sodium bis(trimethylsilyl)amide in THF (7 L) over 1 h, with the temperature maintained <5° C. After 5 min, the reaction was assayed for completion. The reaction was quenched with 1N HCl (22.5 L) and diluted with heptane (20 L). The aqueous layer was cut and the organic layer washed with sat'd. brine (12 L). The solvent was switched to MeOH and water was removed azeotropically until a KF<900 ppm was achieved. The solution of 1-4 was used directly in the next reaction. ethyl (2S,4S)-4-allyl-5-oxo-2,4-diphenyl-1,3-oxazolidine-3-carboxylate: ¹H NMR (CDCl₃, 400 Hz) δ=7.60-7.52 (m, 2H), 7.39-7.33 (m, 8H), 6.55 (m, 1H), 5.84 (m, 1H), 5.38 (m, 2H), 4.16 (m, 2H), 3.72-3.12 (m, 2H), 1.17 (t, J=7.0 Hz, 3H); ¹³C NMR (CDCl₃, 100 MHz): δ=172.5, 164.0, 137.5, 131.0, 130.5, 129.7, 128.3, 128.1, 127.4, 126.2, 122.0, 89.5, 62.0, 42.2, 40.4, 14.2; MS m/z 352 ([M+H]⁺, C₂₁H₂₂NO₄, calc'd 352.15).

### Step 4: methyl (2S)-2-[(ethoxycarbonyl)amino]-2-phenylpent-4-enoate (1-5)

To an 23° C. solution of 1-4 in MeOH (20 L) was added 30% NaOMe in MeOH (535 mL) over 0.25 h, with the temperature maintained <30° C. After 4 h, the reaction was assayed for completion. The reaction was quenched into 5% NaHSO₃ (40 L) and diluted with IPAc (20 L). The aqueous layer was cut and the organic layer washed with 10% KH₂PO₄ (12 L). The solvent was switched to MeCN and used directly in the next reaction. methyl (2S)-2-[(ethoxycarbonyl)amino]-2-phenylpent-4-enoate: ¹H NMR (CDCl₃, 400 Hz) δ=7.46-7.43 (m, 2H), 7.39-7.27 (m, 3H), 6.23 (bs, 1H), 5.76-5.66 (m, 1H), 5.20-5.14 (m, 2M), 4.10-4.00 (m, 2H), 3.68 (s, 3H), 3.53 (bs, 1H), 3.20 (dd, J=13.7, 7.6 Hz, 1H) 1.27-1.15 (m, 3H); ¹³C NMR (CDCl₃, 100 MHz): δ=172.6, 154.3, 139.8, 132.3, 128.4, 127.8, 125.9, 119.4, 65.0, 60.6, 53.1, 37.8, 14.4; MS m/z 300 ([M+Na]⁺, C₁₅H₁₉NNaO₄, calc'd 300.12).

### Step 5: methyl 4-[(ethoxycarbonyl)oxy]-2-phenyl-D-prolinate (1-6)

To a 23° C. solution of 1-5 in MeCN (42 L) was added water (12 L), followed by 12 (8 kg). After 6 h, the reaction was assayed for completion. The reaction was quenched with 10% Na₂SO₃ (35 L), basified with 50 wt % NaOH (4 L) and extracted with IPAc (35 L). The aqueous layer was cut and discarded and the organic layer was extracted with 6N HCl (35 L). The organic layer was discarded. The aq. layer was cooled to −10° C., IPAc (35 L) was added, and slowly neutralized with 22 L of 10N NaOH. The aqueous layer was cut and discarded and the solution of 1-6 was stored. methyl 4-[(ethoxycarbonyl)oxy]-2-phenyl-D-prolinate: ¹H NMR (CDCl₃, 400 Hz) indicated a 2:1 mixture of diastereomers: δ=7.55-7.47 (m, 5H), 7.34-7.22 (m, 5H), 5.18-5.11 (m, 2H), 4.22-4.11 (m, 4H), 3.68 (s, 6H), 3.33-3.24 (m, 4H), 3.10 (d, J=14.1 Hz, 2H), 3.05 (b, 2H), 2.34 (dd J=14.3, 5.5 Hz, 1H), 2.22 (dd J=14.3, 4.1 Hz, 1H), 1.31-1.23 (m, 6H); ¹³C NMR (CDCl₃, 100 MHz): δ=175.2, 175.1, 154.7, 154.4, 142.0, 141.5, 128.3, 128.2, 127.5, 127.4, 126.0, 125.7, 78.5, 77.6, 71.7, 71.0, 63.8, 52.9, 52.8, 52.7, 52.0, 51.8, 43.2, 42.9, 14.1, 14.0; MS m/z 294 ([M+H]⁺, C₁₅H₂₀NO₅, calc'd 294.13).

### Step 6: (5S)-5-(hydroxymethyl)-5-phenylpyrrolidin-3-ol (1-7)

To a solution of carbonate 1-6 (5.0 g, 17.0 mmol) in THF (50 mL) was added Red-Al 3.5M solution in toluene (17.0 mL, 59.7 mmol, 3.5 moleq.) at −50° C. The reaction mixture was warmed up to rt and aged for 2 h. The reaction was quenched by 2.0M Rochelle salt solution (45 mL, ca. 1.5 moleq to Red-Al) at 0° and aged vigorously over 5 h at rt. After the aqueous phase was separated, the mixed organic solution was switched to n-BuOAc by azeotropic distillation under reduced pressure (ca. 20 torr, 60° C.). After 200 mL of n-BuOAc was added, THF, toluene and methoxy ethanol were detected less than 0.1% in GC and KF showed 0.11%. MS m/z 194 ([M+H]⁺, C₁₁H₁₅NO₂, calc'd 193.11).

### Step 7: (7aS)-6-hydroxy-7a-phenyltetrahydro-1H-pyrrolo[1,2-c][1,3]oxazol-3-one (1-8)

To the n-BuOAc solution described in Step 6 was added CDI (3.46 g, 21.3 mmol, 1.25 moleq.) portionwise and aging for 1 h at rt. 30 mL of 2N HCl solution was added to the reaction mixture and aging for 1 h. The aqueous phase was separated and extracted with 30 mL of n-BuOAc after addition of 6.0 g of NaCl. To the combined organic layer was added 150 mg of activated carbon (Darco KB) and the mixture aged overnight. The carbon was filtered through a pad of Solka-Floc. Data: ¹H-NMR (400 MHz, CDCl₃) δ 7.47-7.28 (m, 7H), 4.65 (d, J=8.3 Hz, 1H), 4.64-4.59 (m, 0.4H), 4.57-4.51 (m, 1H), 4.51 (d, J=8.8 Hz, 0.4H), 4.33 (d, J=8.3 Hz, 1H), 4.28 (dd, J=13.1, 6.7 Hz, 0.4H), 4.15 (d, J=8.8 Hz, 0.4H), 3.92 (d, J=12.7 Hz, 1H), 3.28 (dd, J=12.7, 3.9 Hz, 1H), 3.18 (dd, J=13.1, 2.7 Hz, 0.4H), 2.63 (d, J=13.6 Hz, 0.4H), 2.50 (dd, J=13.7, 5.1 Hz, 1H), 2.40 (brd, J=13.7 Hz, 1H), 2.25 (dd, J=13.6, 6.8 Hz, 0.4H).

### Step 8: (7aS)-7a-phenyldihydro-1H-pyrrolo[1,2-c][1,3]oxazole-3,6(5H)-dione (1-9)

I-8 (crude, a portion of above solution 1.40 g assay, 6.38 mmol) in n-BuOAc was concentrated under reduced pressure and 14 mL of MeCN was added to the crude crystals. The solvent ratio was n-BuOAc:MeCN=8:92 in GC. To this solution was added AcOH (1.10 mL, 19.2 mmol, 3.0 moleq.), TPAP (33.6 mg, 0.095 mmol, 1.5 mol %) and 2.0M solution of NaOCl (9.5 mL, 19.2 mmol, 3.0 moleq.) dropwise over 30 nm in at rt. (ca. 5% of chlorinated product was seen in HPLC.) After 30 min, the reaction mixture was diluted with 12 mL of AcOEt and the aqueous phase was separated. The organic phase washed with sat. Na₂S₂O₃aq. and brine. The organic solvent was switched to MTBE and the resulted precipitate was filtered and washed with MTBE. Obtained ketone 1-9; 78% (1.08 g, 4.97 mmol, 99.4 area %, 97.0 w/w %, 0.5 area % of chlorinated product).

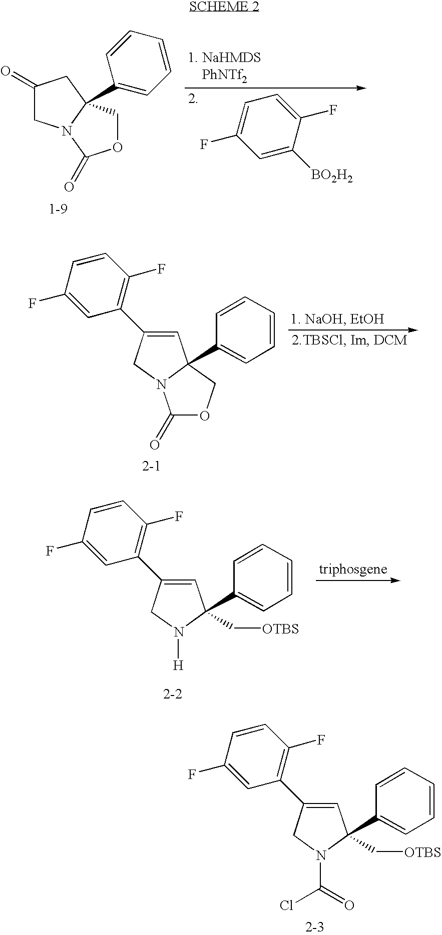

### Step 1: 6-(2,5-Difluorophenyl)-7a(R)-phenyl-5,7a-dihydro-1H-pyrrolo[1,2-c][1,3]oxazol-3-one (2-1)

To a suspension of 2.2 g (10 mmol) of 1-9 in 150 mL of THF at −78° C. is added dropwise 12.2 mL (12.2 mmol) of a 1M solution of NaHMDS in THF. After stirring for 30 min, the solution is allowed to warm to 0° C. and held there for 1 h. The solution is then cooled back down to −78° C. and a solution of 4.35 g (12.2 mmol) of N-phenylbis(trifluoro-methanesulphonimide) in 10 mL of THF is added. The cooling bath was removed and the mixture was allowed to warm to room temperature and stir overnight. The mixture is quenched with a saturated NH₄Cl solution, extracted twice with EtOAc, washed twice with brine, dried over Na₂SO₄ and concentrated. The residue is dissolved in 75 mL of DME and 18 mL of water. To this mixture is added 1.29 g (30 mmol) of LiCl, 3.2 g (30 mmol) of Na₂CO₃, and 4.8 g (30 mmol) of 2,5-difluorophenylboronic acid. The solution is then degassed with N₂ for 1 minute, followed by the addition of 630 mg (0.5 mmol) of tetrakis(triphenylphosphine) palladium (0). The reaction is heated at 90° C. for 3 h, cooled to room temperature, diluted with saturated NaHCO₃, and extracted twice with EtOAc. The combined organic layers are washed with brine, dried over Na₂SO₄, concentrated, and the residue purified by silica gel chromatography with CH₂Cl₂/hexanes to provide 2-1.

### Step 2: 2-({[tert-Butyl(dimethyl)silyl]oxy}methyl)-4-(2,5-difluorophenyl)-2-phenyl-2,5-dihydro-1H-pyrrole (2-2)

A suspension of 1.75 g (5.6 mmol) 2-1 in 15 μL of EtOH and 10 mL of 3 M NaOH is heated at 60° C. for 3 h, then cooled to room temperature. The reaction mixture is combined with a mixture of EtOAc and brine. The layers are separated, the aqueous phase is extracted with EtOAc. The combined organic phases are washed twice with brine, dried over Na₂SO₄, and concentrated to provide a white solid. To this flask is added 30 mL of CH₂Cl₂, 1.5 g (22.3 mmol) of imidazole and 1.76 g (11.7 mmol) of TBSCl, and the resultant suspension was stirred overnight. The reaction is diluted with CH₂Cl₂, washed twice with water, dried over Na₂SO₄, concentrated, and the residue is purified by silica gel chromatography with EtOAc/hexanes to provide 2-2.

### Step 3: (2S)-2-({[tert-Butyl(dimethyl)silyl]oxy}methyl)-4-(2,5-difluorophenyl)-2-phenyl-2,5-dihydro-1H-pyrrole-1-carbonyl chloride 2-3

In a flask equipped with overhead stirrer, thermocouple, and nitrogen/vacuum inlet was charged the S-TBS pyrroline solid 2-2 (180 gms) and IPAC added (1.26 L). Stirring was continued until the solution became homogeneous, about 30 minutes.

In a separate flask equipped with overhead stirrer, thermocouple, and nitrogen/vacuum inlet IPAC added (1.26 L) and the solution cooled to −5° C. Triphosgene was added (67 gms) and then lutidine (173 ml) slowly added. The solution of the S-TBS pyrroline was then added to this solution slowly. The reaction was monitored by HPLC and was considered complete when the conversion of the amine to the product is >99A % at 200 nm by HPLC. The reaction was quenched by adding 1.8 L of 10 wt % aq. citric acid to the reaction mixture. The layers were separated and the organic layer washed twice with water (240 mL). The organic layer was then concentrated to 900 ml (water content was 105 μg/ml) and used directly in the coupling reactions. HPLC assay showed 99.96% conversion to the carbamyl chloride.

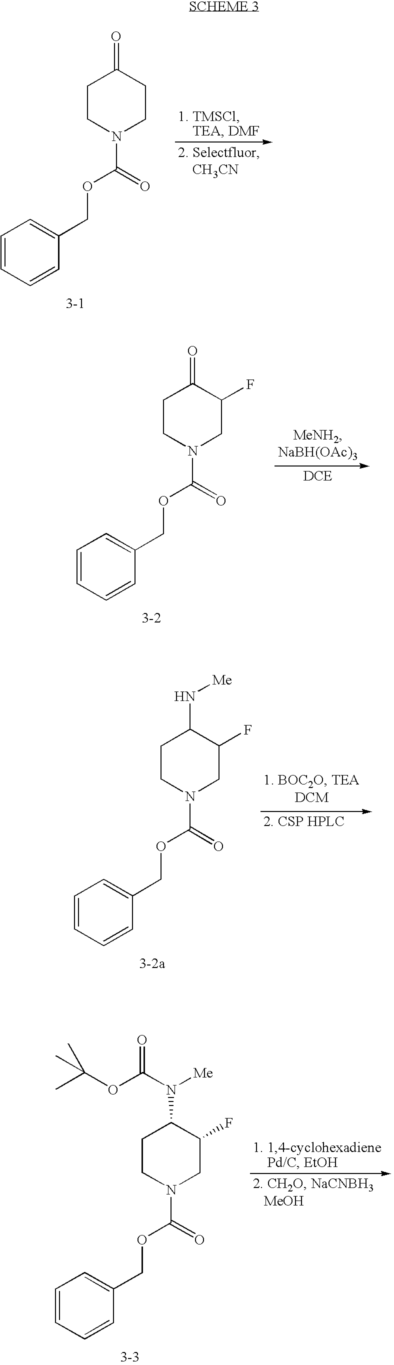

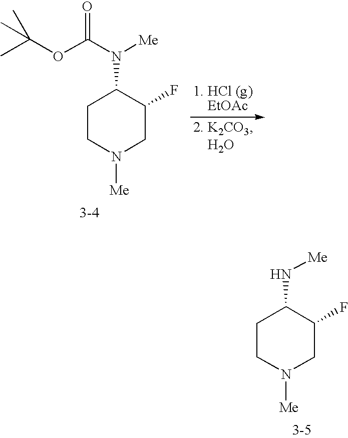

### Step 1: Benzyl 3-fluoro-4-oxopiperidine-1-carboxylate (3-2)

To a solution of 10.0 g (43 mmol) of benzyl-4-oxo-1-piperidinecarboxylate in 25 mL of DMF was added 14.3 mL (103 mmol) of triethylamine and then 6.53 mL (52 mmol) of TMSCl. The reaction was heated at 80° C. overnight, cooled to room temperature, and then dumped into hexanes in a separatory funnel. The mixture was partitioned with saturated aqueous NaHCO₃, separated, washed with brine, dried over MgSO₄ and concentrated by rotary evaporation. The residue was dissolved in 500 mL of CH₃CN and treated with 16.7 g (47 mmol) of Selectfluor. After 90 min the reaction was concentrated to about half the original volume, partitioned between EtOAc and brine, separated, dried over MgSO₄, filtered, and concentrated by rotary evaporation. The residue was loaded onto a silica gel column and eluted with EtOAc/hexanes to provide 3-2 as a colorless oil.

### Step 2: Benzyl 3-fluoro-4-(methylamino)piperidine-1-carboxylate (3-2a)

To a solution of 9.4 g (37.5 mmol) of 3-2 in 150 mL of 1,2-dichloroethane was added 37.5 mL (74.9 mmol) of a 2M solution of methylamine in THF and 11.9 g (56.2 mmol) of Na(OAc)₃BH. After stirring for 2 h, the reaction was quenched with saturated aqueous K₂CO₃, partitioned with EtOAc, separated, and the aqueous phase extracted 3×EtOAc. The combined organic extracts were washed with brine, dried over MgSO₄, filtered, and concentrated by rotary evaporation. The residue was loaded onto a silica gel column and eluted with 80:10:10 CHCl₃/EtOAc/MeOH to provide both the cis and trans isomers of 3-2a as colorless oils. Data for the trans isomer of 3-2a, first to elute (confirmed by NOE analysis): ¹HNMR (600 MHz, CD₂Cl₂) δ 7.4-7.3 (m, 5H), 5.1 (m, 2H), 4.4-4.1 (m, 2H), 3.9 (m, 1H), 3.15-3.05 (m, 2H), 2.75 (m, 1H), 2.4 (s, 3H), 2.0 (m, 1H), 1.25 (m, 1H) ppm. Data for the cis isomer of 2-2a, second to elute (confirmed by NOE analysis): ¹HNMR (600 MHz, CD₂Cl₂) δ 7.4-7.2 (m, 5H), 5.1 (m, 2H), 4.9-4.7 (m 1H), 4.4 (m, 1H), 4.15 (m, 1H), 3.1-2.9 (m, 2H), 2.6 (m, 1H), 2.4 (s, 3H), 1.8 (m, 1H), 1.6 (m, 1H) ppm. HRMS (ES) calc'd M+H for C₁₄H₁₉F₁N₂O₂: 267.1504. Found: 267.1500.

### Step 3: Benzyl (3R,4S)-4-[(tert-butoxycarbonyl)(methyl)amino]-3-fluoropiperidine-1-carboxylate (3-3)

To a solution of 7.67 g (28.8 mmol) of cis-3-2a in 150 mL of CH₂Cl₂ was added 12.1 mL (86.5 mmol) of triethylamine and 9.44 g (43.3 mmol) of di-tert-butyl dicarbonate. After stirring for 1 h, the reaction was partitioned between CH₂Cl₂ and H₂O, the organic phase washed with brine, dried over MgSO₄, filtered and concentrated by rotary evaporation. The residue was loaded onto a silica gel column and eluted with EtOAc/hexanes to provide racemic cis-3-3 as a white solid. Resolution of the enantiomers was carried out chromatographically using a Chiralpak AD© 10×50 cm column with 20% isopropanol in hexanes (with 0.1% diethylamine) at 150 L/min. Analytical HPLC analysis of the eluent on a 4×250 mm Chiralpak AD© column with 20% isopropanol in hexanes (with 0.1% diethylamine) at 1 mL/min indicated that first eluting enantiomer (enantiomer of 3-3) has Rₜ=5.90 min and the second enantiomer (3-3) has Rₜ=6.74 min. Data for 3-3: HRMS (ES) calc'd M+Na for C₁₉H₂₇F₁N₂O₄: 389.1847. Found: 389.1852.

### Step 4: tert-Butyl [(3R,4S)-3-fluoro-1-methylpiperidin-4-yl]methylcarbamate (3-4)

To a solution of 4.6 g (12.6 mmol) of the second eluting enantiomer 3-3 in 150 mL of EtOH was added 29.7 mL (314 mmol) of 1,4-cyclohexadiene and a catalytic amount of 10% Pd on carbon. After stirring overnight, the reaction was filtered through Celite, and concentrated by rotary evaporation. The residue was dissolved in 75 mL of MeOH, 2 mL of AcOH and 3.1 mL (38 mmol) of 37% aqueous formaldehyde were added, and the mixture was stirred for 1 h. At that time, 1.58 g (25.1 mmol) of NaCNBH₃ in 10 mL of MeOH was added and the reaction was aged for 2 h more before being dumped into saturated aqueous NaHCO₃. After extracting with 3×CH₂Cl₂, the organic phase was washed with water, dried over MgSO₄, filtered, and concentrated by rotary evaporation to provide 3-4 as a colorless oil. Data for 3-4: HRMS (ES) calc'd M+H for C₁₂H₂₃FN₂O₂: 247.1817. Found: 247.1810.

### Step 5: (3R,4S)-3-Fluoro-N,1-dimethylpiperidin-4-amine (3-5)

To a solution of 3.0 g (12.2 mmol) of 3-4 in 100 mL of EtOAc was bubbled HCl gas until the solution was warm to the touch. The flask was then capped and stirred for 4 h. The volatiles were removed by rotary evaporation, and the residue was triturated with Et₂O and placed under high vacuum to provide a white solid. This material was mixed with 25 mL of 15% aqueous Na₂CO₃ and extracted with 5×50 mL 2:1 CHCl₃/EtOH. The organic was concentrated by rotary evaporation with very mild heating, the residue was dissolved in 200 mL of CHCl₃, dried over Na₂SO₄, and concentrated to provide 3-5 as a colorless oil. Data for 3-5: ¹HNMR (500 MHz, CDCl₃) δ 4.8 (m, 1H), 3.15 (m, 1H), 2.85 (m, 1H), 2.5 (s, 3H), 2.45 (m, 1H), 2.3 (s, 3H), 2.2-2.0 (m, 2H), 1.9-1.7 (m, 2H) ppm. HRMS (ES) calc'd M+H for C₇H₁₅FN₂: 147.1292. Found: 147.1300.

### Scheme 3A

### Step 1: Benzyl 3-fluoro-4-oxopiperidine-1-carboxylate (3-2)

A 22-L round bottom flask with mechanical stirrer was charged with Cbz-ketone 3-1 (2.5 kg, 10.7 mol), 5.0 L of dimethylacetamide, triethylamine (3.0 L, 21.5 mol). Trimethylsilylchloride (2.0 L, 15.7 mol) was added. The mixture heated to 60° C. and aged for 4 hours. After cooling to 10° C., the mixture was quenched into 10 L of 5% sodium bicarbonate and 10 L n-heptane maintaining the internal temperature at less than 20° C. The organic layer washed twice with 10 L of 2.5% sodium bicarbonate. The final organic layer was dried over sodium sulfate, filtered, and concentrated under reduced pressure and solvent switched to 10 L MeCN.

A 50-L jacketed vessel was charged with 7.5 L of MeCN and Selectfluor (4.1 kg, 11.5 mol). The slurry was cooled to 10° C. and potassium carbonate (0.37 kg, 2.68 mol) added. The silyl ether solution in MeCN was transferred in portions maintaining the internal temperature at 10-15° C. The final slurry was aged for 2 hours at 10-15° C. The reaction was quenched into a 100 L extractor containing 20 L of 2 N hydrochloric acid and 30 L of ethyl acetate. The organic layer washed with 20 L of 2 N hydrochloric acid, 10 L of 20 wt % sodium chloride, dried over sodium sulfate, and filtered. The filtrate was concentrated and flushed with dry EtOAc under reduced pressure to KF=16000 μg/mL and then solvent switch under reduced pressure to ˜10 L THF.

### Step 2: Benzyl 3-fluoro-4-(methylamino)piperidine-1-carboxylate (3-2a)

In a round-bottom flask, Cbz fluoroketone (10.3 mol) was dissolved in tetrahydrofuran (30 L). Methylamine, 2 M in tetrahydrofuran (2.00 equiv; 20.6 moles; 10.3 L) was added and the mixture stirred for 30 min at room temp. The mixture was cooled to 0° C. and acetic acid (20.6 moles; 1.17 L; 1.236 kg) added followed by stirring at 0° C. for another 30 minutes. Sodium triacetoxyborohydride (12.36 moles; 2.62 kg) was added in portions to the solution in 15 minutes and the reaction mixture was aged at 0° C. until completion as judged by HPLC analysis.

The reaction mixture was transferred slowly into a 100 L cylindrical extractor containing hydrochloric acid, 12 M in water (30.9 moles, 2.575 L), water (30 L), and toluene (140 mol, 15 L). After vigorous stirring for 15 minutes, the layers were separated and toluene layer further washed with water (10 L). The combined aqueous layer was transferred back into the extractor. Sodium hydroxide, 10 M in water (82.4 mole, 8.24 L), was added and the mixture extracted once with IPAC (30 L).

The organic layer was dried with sodium sulfate (3 kg) and concentrated. The residue was dissolved in 8:2 (vol:vol) ethanol:water (23 kg ethanol mixed with 7.2 kg water), 85% phosphoric acid (9.83 mol, 952 g, 667 mL) was added to the solution and crystal seeds were added. The mixture was stirred at room temperature overnight. Crystalline solid precipitated and was collected by filtration. The solid washed with 8:2 ethanol:water and dried in vacuum oven to give 2.1 kg solid.

The solid was suspended in 36 L EtOH and 4 L water mixture and the mixture was heated to 70° C.-80° C. until all solid dissolved. The heat source was removed and the clear solution was seeded with the cis isomer mixture 3-2a. After stirring at room temperature overnight, a crystalline solid precipitated and was collected by filtration. The solid product was dried in vacuum oven to give white solid.

### Step 3: Benzyl (3R,4S)-4-[(tert-butoxycarbonyl)(methyl)amino]-3-fluoropiperidine-1-carboxylate 3-3

In a 50 L extractor was charged 20 L water and 1.06 kg Na₂CO₃, the mixture was stirred until all solid was dissolved. IPAC (20 L) and CBZ amine phosphate (1.85 kg, 5.3 mol) were added. The layers were cut after mixing. The aqueous layer was extracted with another 5 L IPAC. The combined organic layers were dried with sodium sulfate. After the drying agent was filtered off, the batch was charged into a 72 L round bottom flask, and Boc₂O solution (1.0 M, 4.8 L) was added. HPLC assay after 45 min indicated 98% conversion. Additional Boc₂O solution (50 mL) was added. After the batch was aged for additional 15 hours, it was concentrated under vacuum to the minimum volume, flushed with MeOH (10 L-15 L). The batch was diluted with methanol to a total weight of ca. 14.3 kg. HPLC assay indicated ca. 1.9 kg desired product.

The fluoropiperidine was resolved by chromatographic separation on 20 micron Chiralpak AD (Diacel Chemical Industries, Ltd.) chiral stationary phase column (30 ID×25 cm). An amount of 54 g of racemate per injection was eluted with methanol. The lowest retention time enantiomer was collected giving 45 g (85% recovery) of the desired (3R,4S) enantiomer in 98% ee. This separation process was repeated and the desired fractions from different injections were combined and concentrated.

### Step 4: tert-Butyl [(3R,4S)-3-fluoro-1-methylpiperidin-4-yl]methylcarbamate (3-4)

The concentrated solution (4 L) from the chiral separation step was shown to contain 489.5 g (1.3 mol) of Cbz-Boc-diamine 3-3. To this solution, formaldehyde (37% in water, 430 mL, 5.3 mol) was added and the mixture pressurized under hydrogenated over 5% Pd/C (183 g) for 4 hours. The reaction mixture was filtered to remove the catalyst and partitioned between 8 L of EtOAc and 8 L of 0.5 M sodium bicarbonate. The organic layer washed with 8 L of 0.5 M sodium bicarbonate. The combined aqueous layers were back extracted with 8 L of EtOAc. The combined organic layers were dried over sodium sulfate and filtered. The filtrate was used in the next step directly.

### Step 6: (3R,4S)-3-Fluoro-N,1-dimethylpiperidin-4-amine (3-5)

The ethyl acetate solution containing the Boc protected diamine 3-4 (327 g by HPLC assay) was charged to a 12 L flask while concentrating at 28° C. When the batch had a total volume of 1.5 L, the batch was then solvent switched to ethanol by charging 8 L of ethanol while distilling at a constant volume.

To a different 12 L round bottom flask was added 1.5 L of ethanol (200 proof, punctilious). 436 mL of acetyl chloride was then added to the ethanol maintaining the temperature below 35° C. with the aid of a water bath. The solution was stirred for 1 h. The ethanol solution containing 302 g of the Boc protected diamine 2-4 was then slowly added to the ethanolic HCl (AcCl+EtOH) solution, maintaining temp <30° C. At the point where ¾ of the addition was complete, solids began to crystallize from the solution. The reaction was monitored by GC and the slurry stirred overnight. The solids were isolated by filtration and cake washed with 2 L of 85% ethanol, 15% ethyl acetate. The filter cake was then dried under vacuum with a stream of N₂ overnight to yield 243 g of the desired product 3-5 as a dihydrochloride salt (Form 1). GC analysis indicated the batch to be 99.3% ee.

Thermal Analysis

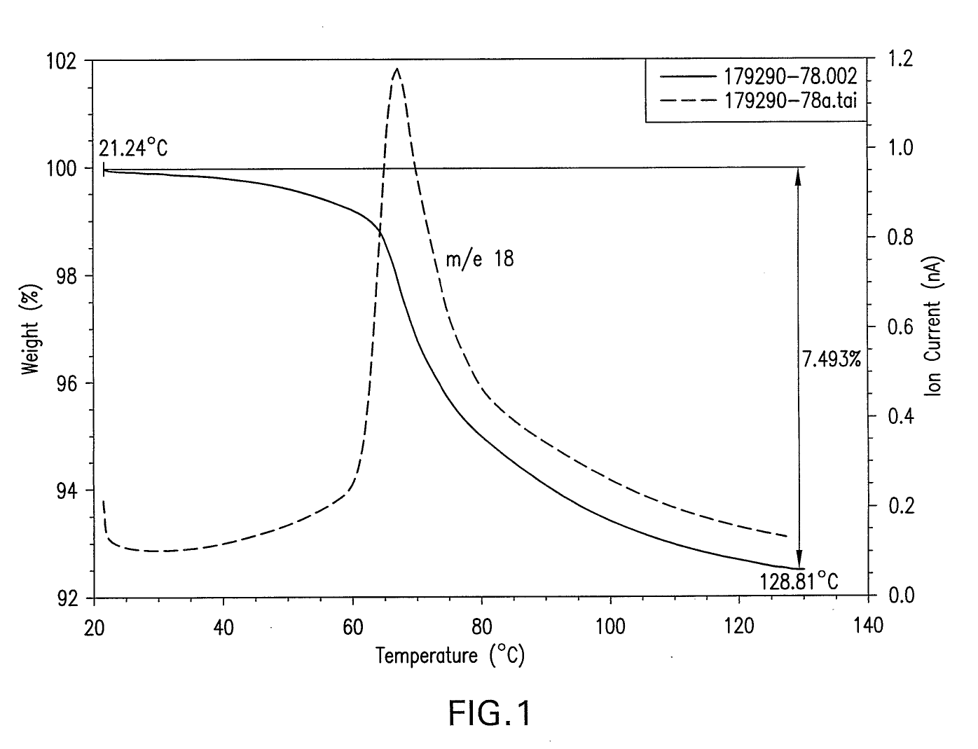

TG-MS of the diamine dihydrochloride salt sample of 3-5 (Form 1) produced the weight loss curve shown in FIG. 1 (solid line). The MS data indicate that the 7.5% weight loss is associated the evolution of approximately one mole of water (dashed line). The theoretical weight loss for a monohydrate is 7.6%. The instrument used to collect this data is a TA instruments TGA Q500 attached to a Pfeiffer quadrupole mass analyzer. A scan rate of 10° C./min was used.

XRPD

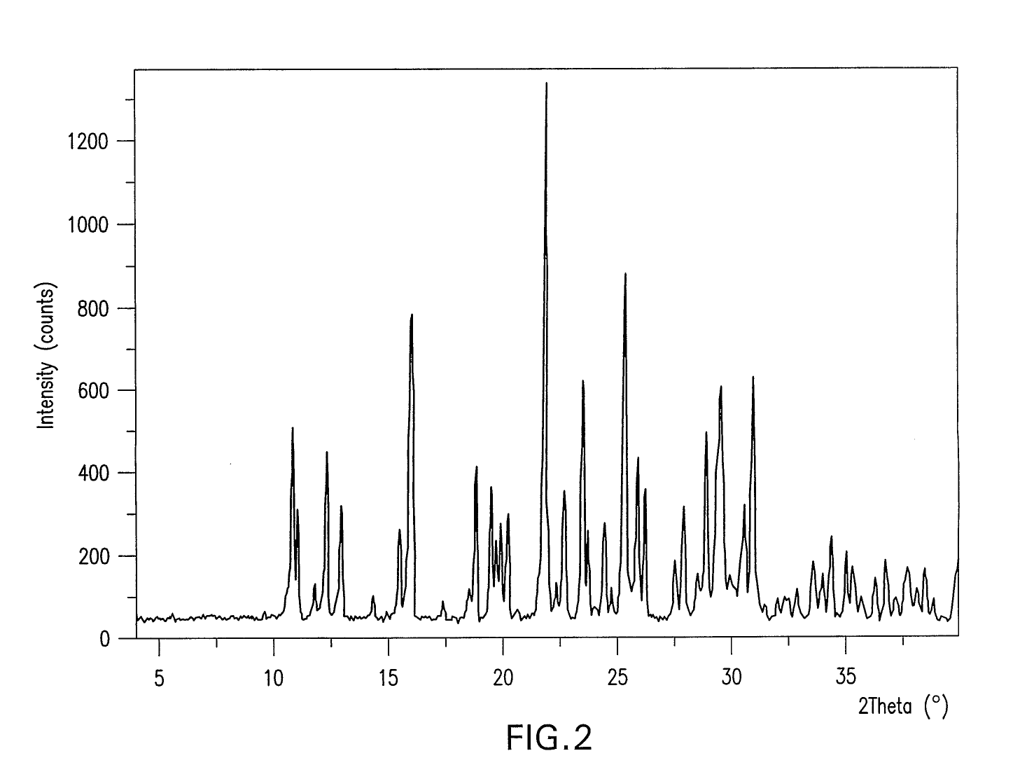

The X-ray powder diffraction (Cu K alpha radiation) of the diamine dihydrochloride salt sample of 3-5 dihydrochloride salt, Form 1) produced the powder diffraction pattern shown in FIG. 2. The characteristic peaks and key d-spacings listed below in the Table 1. The pattern was acquired on a Philips analytical x-ray from 4° to 40° (20) using the spinning stage over approx. 8 min.

Key d-Spacings.

TABLE 1
2Theta values and observed relative intensities

| 2Theta | d-spacing | I_{obs}. |
|---|---|---|
| 10.9 | 8.2 | 307 |
| 12.4 | 7.2 | 305 |
| 16.0 | 5.5 | 936 |
| 18.9 | 4.7 | 325 |
| 21.9 | 4.0 | 1000 |
| 23.6 | 3.8 | 495 |
| 25.5 | 3.5 | 746 |
| 26.0 | 3.4 | 427 |
| 29.0 | 3.1 | 349 |
| 29.6 | 3.0 | 376 |
| 31.0 | 2.9 | 516 |

Approximately 200 mg of 3-5 (fluoropiperidine 2HCl salt) was added to a vial and suspended in methanol (<500 μL). The sample was heated to dissolution with a heat gun. After 2 hours large 3 dimensional crystals were noted. Crystals of 3-5 (fluoropiperidine 2HCl salt Form 2) were isolated by removal of the remaining solvent.

A single crystal of 3-5 (fluoropiperidine 2HCl salt Form 2) was selected for single crystal x-ray data collection on a Bruker Smart Apex system. The crystal was colorless plate with dimensions of 0.24 mm×0.22 mm×0.14 mm. The unit cell was collected on 5 second scan rate and auto indexing gave the cell setting to be monoclinic. The structure was solved in the monoclinic P 2₁ space group after a quadrant data collection using 5 second scan rate. The 3-5 structure was determined to be the structure having the absolute stereochemistry shown in above scheme.

Singel crystal X-ray diffraction data and structure refinement for 3-5 (Form 2).

| | |
|---|---|
| Empirical formula | C₈H₂₁C₁₂FN₂O |
| Formula weight | 251.17 |
| Temperature | 298(2) K |
| Wavelength | 0.71073 A |
| Crystal system, space group | Monoclinic, P2(1) |
| Unit cell dimensions: | a = 7.286(2) Å alpha = 90 deg. |
| | b = 7.637(2) Å beta = 105.295(5) deg. |
| | c = 12.378(4) Å gamma = 90 deg. |
| Volume | 664.3(4) Å³ |
| Z, Calculated density | 2, 1.256 Mg/m³ |
| Absorption coefficient | 0.477 mm⁻¹ |
| F(000) | 268 |
| Crystal size | 0.24 × 0.22 × 0.14 mm |
| Theta range for data collection | 1.71 to 26.35 deg. |
| Limiting indices | -9 < = h < = 9, -9 < = k < = 9, |
| | -15 < = l < = 15 |
| Reflections collected/unique | 5309/2674 [R(int) = 0.0227] |
| Completeness to theta = 26.35 | 99.7% |
| Absorption correction | None |
| Refinement method | Full-matrix least-squares on F² |
| Data/restraints/parameters | 2674/1/135 |
| Goodness-of-fit on F² | 1.055 |
| Final R indices [I > 2sigma(I)] | R1 = 0.0383, wR2 = 0.0939 |
| R indices (all data) | R1 = 0.0409, wR2 = 0.0959 |
| Absolute structure parameter | 0.02(6) |
| Largest diff. peak and hole | 0.310 and -0.135 e.Å⁻³ |

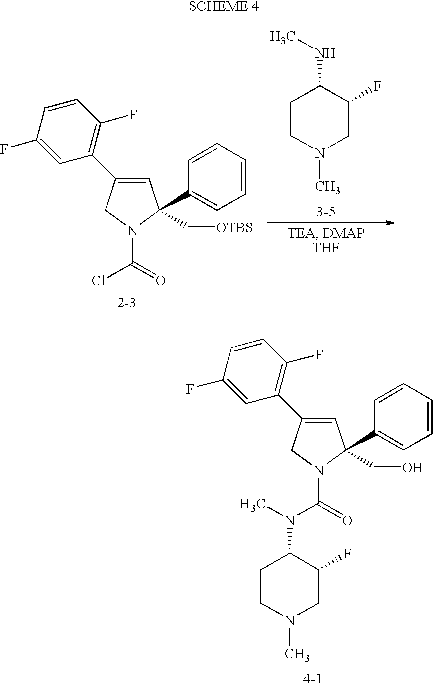

### (2S)-4-(2,5-Difluorophenyl)-N-[(3R,4S)-3-fluoro-1-methylpiperidin-4-yl]-2-(hydroxymethyl)-N-methyl-2-phenyl-2,5-dihydro-1H-pyrrole-1-carboxamide (4-1)

In a flask equipped with overhead stirrer, thermocouple, and nitrogen/vacuum inlet was charged the carbamyl chloride 2-3 in IPAC (0.9 L). To this solution was added 0.9 L DMF, 111 gms fluorodiamine 3-5 and 540 ml diisopropylethylamine. The solution was warmed to 60° C. for 15 hrs and assayed for conversion of carbamyl chloride to product. The reaction is considered complete when the conversion of carbamyl chloride to product is >98A % at 200 nm by HPLC. The reaction was cooled to 5° C. and 450 ml 6NHCl was added. The solution was aged until desilylation was complete (>99A % at 200 nm), about 2 hrs.

Isopropylacetate (3 L) and then 8 wt % aqueous sodium bicarbonate was added (2 L) to the reaction mixture, which was allowed to warm to 15-20° C. The layers were separated and the aqueous layer extracted once with 3 L IPAC. The combined organic layers were washed twice with 1 L water. The washed organic solution was concentrated to 5 liters and, while at 35-40° C. transferred to another flask through a 1 um polypropylene filter. Distillation was continued until a volume of 1 L was obtained and then the reaction was cooled to room temperature over two hours. Heptane (1 L) was then slowly added over 2 hrs. The resultant slurry was filtered onto a sintered glass funnel and the crystalline product was washed 3 times with 500 mls of 2:1 heptane:isopropylacetate as displacement washes. The solid 4-1 was dried with a sweep of nitrogen overnight.

A single crystal from the above preparation was selected for single crystal x-ray data collection on a Bruker Smart Apex system. The crystal was colorless polyhedron with dimensions of 0.14 mm×0.13 mm×0.13 mm. The unit cell was collected on 30 second scan rate and auto indexing gave the cell setting to be orthorhombic. The structure was solved in the orthorhombic P 2₁ 2₁ 2₁ space group after a quadrant data collection using 30 second scan rate. The relative and absolute stereochemistry of 4-1 based on the X-ray structural determination for compounds 4-1 and 3-5 is shown in the above scheme.

1. A process for the preparation of a compound of Formula I: 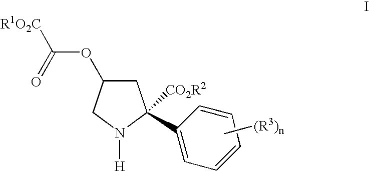
or a salt thereof,
wherein:
a is 0 or 1;
b is 0 or 1;
m is 0, 1, or 2;
n is 1 or 2;
r is 0 or 1;
s is 0 or 1;
R¹ and R² are independently selected from: (C₁-C₆)alkyl, aryl, heterocyclyl and (C₃-C₆)cycloalkyl, optionally substituted with one, two or three substituents selected from R⁴;
R³ is selected from:
1) hydrogen;
2) (C═O)ₐO_{b}C₁-C₁₀ alkyl,
3) (C═O)ₐO_{b}aryl,
4) CO₂H,
5) halo,
6) CN,
7) OH,
8) O_{b}C₁-C₆ perfluoroalkyl,
9) Oₐ(C═O)_{b}NR⁵R⁶,
10) S(O)ₘR^{a},
11) S(O)₂NR⁵R⁶, and
12) —OPO(OH)₂;
said alkyl and aryl, optionally substituted with one, two or three substituents selected from R⁴;
R⁴ is selected from:
1) (C═O)ᵣOₛ(C₁-C₁₀)alkyl,
2) Oᵣ(C₁-C₃)perfluoroalkyl,
3) oxo,
4) OH,
5) halo,
6) CN,
7) (C₂-C₁₀)alkenyl,
8) (C₂-C₁₀)alkynyl,
9) (C═O)ᵣOₛ(C₃-C₆)cycloalkyl,
10) (C═O)ᵣOₛ(C₀-C₆)alkylene-aryl,
11) (C═O)ᵣOₛ(C₀-C₆)alkylene-heterocyclyl,
12) (C═O)ᵣOₛ(C₀-C₆)alkylene-N(R^{b})₂,
13) C(O)R^{a},
14) (C₀-C₆)alkylene-CO₂R^{a},
15) C(O)H,
16) (C₀-C₆)alkylene-CO₂H, and
17) (C═O)ᵣN(R^{b})₂,
18) S(O)ₘR^{a},
19) S(O)₂N(R^{b})₂; and
20) —OPO(OH)₂;
said alkyl, alkenyl, alkynyl, cycloalkyl, aryl, alkylene and heterocyclyl is optionally substituted with up to three substituents selected from R^{b}, OH, (C₁-C₆)alkoxy, halogen, CO₂H, CN, O(C═O)C₁-C₆ alkyl, oxo, NO₂ and N(R^{b})₂;
R⁵ and R⁶ are independently selected from:
1) H,
2) (C═O)O_{b}C₁-C₁₀ alkyl,
3) (C═O)O_{b}C₃-C₈ cycloalkyl,
4) (C═O)O_{b}aryl,
5) (C═O)O_{b}heterocyclyl,
6) C₁-C₁₀ alkyl,
7) aryl,
8) C₂-C₁₀ alkenyl,
9) C₂-C₁₀ alkynyl,
10) heterocyclyl,
11) C₃-C₈ cycloalkyl,
12) SO₂R^{a}, and
13) (C═O)NR^{b}₂,
said alkyl, cycloalkyl, aryl, heterocylyl, alkenyl, and alkynyl is optionally substituted with one, two or three substituents selected from R⁴, or
R⁵ and R⁶ can be taken together with the nitrogen to which they are attached to form a monocyclic or bicyclic heterocycle with 3-7 members in each ring and optionally containing, in addition to the nitrogen, one or two additional heteroatoms selected from N, O and S, said monocyclic or bicyclic heterocycle optionally substituted with one, two or three substituents selected from R⁴;
R^{a} is independently selected from: (C₁-C₆)alkyl, (C₃-C₆)cycloalkyl, aryl, or heterocyclyl, optionally substituted with one, two or three substituents selected from R⁴;
R^{b} is independently selected from: H, (C₁-C₆)alkyl, aryl, heterocyclyl, (C₃-C₆)cycloalkyl, (C═O)OC₁-C₆ alkyl, (C═O)C₁-C₆ alkyl, (C═O)aryl, (C═O)heterocyclyl, (C═O)NR^{e}R^{e′} or S(O)₂R^{a}, optionally substituted with one, two or three substituents selected from R⁷;
which comprises the step of reacting a compound of the formula II: 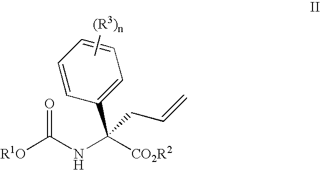
with a halogenating agent in an aqueous solvent to produce the compound of formula I.

2. The process according to claim 1 wherein the halogenating agent is iodine (I₂).

3. The process according to claim 1 wherein R¹ is ethyl, R² is methyl and R³ is hydrogen.

4. The process according to claim 1 for preparing a compound of the formula I which further comprises the steps of
a) reacting the compound of the formula III: 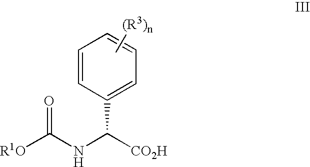
with a benzaldehyde source, in the presence of an acid to produce the compound of the formula IV: 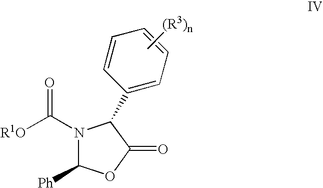
and b) converting the compound of the formula IV to the compound of formula II;
wherein R³ is as described in claim 1.

5. The process according to claim 4, wherein the benzaldehyde source is benzaldehyde dimethylacetal.

6. The process according to claim 4, which further comprises the additional step of crystallizing the compound of formula IV from a crystallization solvent prior to converting the compound of the formula IV to the compound of formula II.

7. The process according to claim 4, wherein the conversion of the compound of the formula IV to the compound of formula II comprises the step of adding a base to a solution of a mixture of the compound of the formula IV and an allylating agent.

8. A process for preparing a compound of the formula V, or a salt thereof: 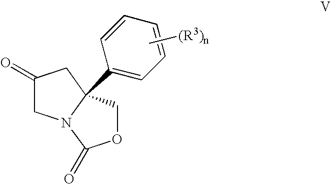
which comprises the steps of:
a) converting the compound of the formula I, 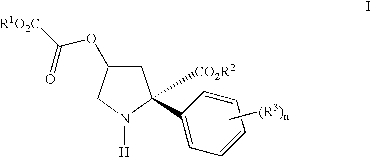
or a salt thereof,
wherein:
a is 0 or 1;
b is 0 or 1;
m is 0, 1, or 2;
n is 1 or 2;
r is 0 or 1;
s is 0 or 1;
R¹ and R² are independently selected from: (C₁-C₆)alkyl, aryl, heterocyclyl and (C₃-C₆)cycloalkyl, optionally substituted with one, two or three substituents selected from R⁴;
R³ is selected from:
1) hydrogen;
2) (C═O)ₐO_{b}C₁-C₁₀ alkyl,
3) (C═O)ₐO_{b}aryl,
4) CO₂H,
5) halo,
6) CN,
7) OH,
8) O_{b}C₁-C₆ perfluoroalkyl,
9) Oₐ(C═O)_{b}NR⁵R⁶,
10) S(O)ₘR^{a},
11) S(O)₂NR⁵R⁶, and
12) —OPO(OH)₂;
said alkyl and aryl, optionally substituted with one, two or three substituents selected from R⁴;
R⁴ is selected from:
1) (C═O)ᵣOₛ(C₁-C₁₀)alkyl,
2) Oᵣ(C₁-C₃)perfluoroalkyl,
3) oxo,
4) OH,
5) halo,
6) CN,
7) (C₂-C₁₀)alkenyl,
8) (C₂-C₁₀)alkynyl,
9) (C═O)ᵣOₛ(C₃-C₆)cycloalkyl,
10) (C═O)ᵣOₛ(C₀-C₆)alkylene-aryl,
11) (C═O)ᵣOₛ(C₀-C₆)alkylene-heterocyclyl,
12) (C═O)ᵣOₛ(C₀-C₆)alkylene-N(R^{b})₂,
13) C(O)R^{a},
14) (C₀-C₆)alkylene-CO₂R^{a},
15) C(O)H,
16) (C₀-C₆)alkylene-CO₂H, and
17) (C═O)ᵣN(R^{b})₂,
18) S(O)ₘR^{a},
19) S(O)₂N(R^{b})₂; and
20) —OPO(OH)₂;
said alkyl, alkenyl, alkynyl, cycloalkyl, aryl, alkylene and heterocyclyl is optionally substituted with up to three substituents selected from R^{b}, OH, (C₁-C₆)alkoxy, halogen, CO₂H, CN, O(C═O)C₁-C₆ alkyl, oxo, NO₂ and N(R^{b})₂;
R⁵ and R⁶ are independently selected from:
1) H,
2) (C═O)O_{b}C₁-C₁₀ alkyl,
3) (C═O)O_{b}C₃-C₈ cycloalkyl,
4) (C═O)O_{b}aryl,
5) (C═O)O_{b}heterocyclyl,
6) C₁-C₁₀ alkyl,
7) aryl,
8) C₂-C₁₀ alkenyl,
9) C₂-C₁₀ alkynyl,
10) heterocyclyl,
11) C₃-C₈ cycloalkyl,
12) SO₂R^{a}, and
13) (C═O)NR^{b}₂,
said alkyl, cycloalkyl, aryl, heterocylyl, alkenyl, and alkynyl is optionally substituted with one, two or three substituents selected from R⁴, or
R⁵ and R⁶ can be taken together with the nitrogen to which they are attached to form a monocyclic or bicyclic heterocycle with 3-7 members in each ring and optionally containing, in addition to the nitrogen, one or two additional heteroatoms selected from N, O and S, said monocyclic or bicyclic heterocycle optionally substituted with one, two or three substituents selected from R⁴;
R^{a} is independently selected from: (C₁-C₆)alkyl, (C₃-C₆)cycloalkyl, aryl, or heterocyclyl, optionally substituted with one, two or three substituents selected from R⁴;
R^{b} is independently selected from: H, (C₁-C₆)alkyl, aryl, heterocyclyl, (C₃-C₆)cycloalkyl, (C═O)OC₁-C₆ alkyl, (C═O)C₁-C₆ alkyl, (C═O)aryl, (C═O)heterocyclyl, (C═O)NR^{e}R^{e′} or S(O)₂R^{a}, optionally substituted with one, two or three substituents selected from R⁷;
to the compound of the formula VI: 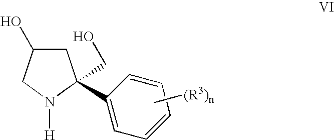
b) reacting the compound of the formula VI with a carbon monoxide diradical source to produce the compound of the formula VII: 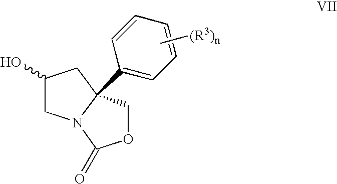
and
c) reacting the compound of the formula VII with an oxidizing agent to product the compound of the formula V.

9. The process according to claim 8 wherein R³ is hydrogen.

10. The process according to claim 8 wherein carbon monoxide diradical source is 1,1′-carbonyldiimidazole.

11. The process according to claim 8 wherein the oxidizing agent is sodium hypochlorite with a catalytic amount of tetrapropylammoniumperruthenate.

12. A compound which is selected from: 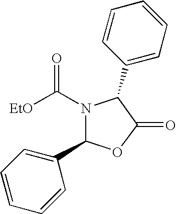
ethyl (2S,4R)-5-oxo-2,4-diphenyl-1,3-oxazolidine-3-carboxylate; 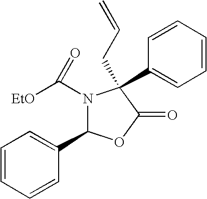
ethyl (2S,4S)-4-allyl-5-oxo-2,4-diphenyl-1,3-oxazolidine-3-carboxylate; 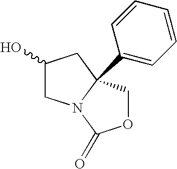
(7aS)-6-hydroxy-7a-phenyltetrahydro-1H-pyrrolo[1,2-c][1,3]oxazol-3-one; and 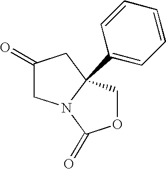
(7aS)-7a-phenyldihydro-1H-pyrrolo[1,2-c][1,3]oxazole-3,6(5H)-dione.

13. (canceled)

14. (canceled)

15. (canceled)

16. A (3R,4S)-3-fluoro-N,1-dimethylpiperidin-4-amine dihydrochloride Form 1 characterized by an X-ray powder diffraction data selected from:
a) X-ray powder diffraction pattern substantially similar to that set forth in FIG. 2; and
b) X-ray (Cu K alpha radiation) powder diffraction pattern including characteristic peaks at about 10.9, 12.4, 16.0, 18.9, 21.9, 23.6, 25.4, 26.0, 29.0, 29.6 and 31.0 degrees 2θ.

17. (canceled)

18. A (3R,4S-3-fluoro-N,1-dimethylpiperidin-4-amine dihydrochloride Form 2 characterized by the following single crystal X-ray diffraction unit cell parameters: a=7.286(2) Å, b=7.637(2) Å, c=12.378(4) Å, alpha=90 deg, beta=105.295(5) deg and gamma=90 deg.
